(19) United States
(12) Patent Application Publication
(10) Pub. No.: US 2011/0230737 A1
(43) Pub. Date: Sep. 22, 2011

(54) DEVICE AND METHOD FOR EARLY DIAGNOSIS AND PROGNOSIS OF HEALING PROGRESSIONS, IN PARTICULAR FOR BONE INJURIES

(75) Inventors: Georg Duda, Berlin (DE); Ulrike Lehnigk, Hamburg (DE); Ansgar Petersen, Berlin (DE); Juliane Gläser, Pracht (DE)

(73) Assignee: CHARITÉ- UNIVERSITÃTSMEDIZIN BERLIN, Berlin (DE)

(21) Appl. No.: 13/121,854

(22) PCT Filed: Oct. 1, 2009
(86) PCT No.: PCT/EP2009/062759

§ 371 Date: Jun. 1, 2011

Related U.S. Application Data
(60) Provisional application No. 61/101,716, filed on Oct. 1, 2008.

(51) Int. Cl. A61B 5/00; A61B 10/00; G01N 1/28

(52) U.S. Cl. 600/309; 600/562; 73/61.59

(57) ABSTRACT

The invention relates to a device comprising: i) a sample-receiving unit (20), said sample-receiving unit (20) comprising a cavity (24) for receiving sample material, means (22) for applying pressure in a regulatable and/or controllable fashion to sample material located in the cavity, and an outlet (25), said outlet (25) being disposed and designed in such a way that, by actuating the means (22) for applying pressure in a regulatable and/or controllable fashion, components of the sample material can be transferred from the cavity (24) through the outlet (25) and directly to an analysis unit (30) connectable to the outlet (25); and ii) an analysis unit (30), said analysis unit (30) comprising an inlet (31), a first filter (32), a reservoir (33) and an analysis track (35), said inlet (31) being designed to be connectable to an outlet (25) of the sample-receiving unit (20) such that components of the sample material can be fed from the sample-receiving unit (20) directly to the analysis unit (30), said first filter (32) being disposed between inlet (31) and reservoir (33), so that substantially liquid components of the sample material can be transferred from the inlet (31) through the filter (32) into the reservoir (33), said reservoir (33) and analysis track (35) being disposed and designed in such a way that filtrate can be transferred from the reservoir (33) into the analysis track (35); wherein the outlet (25) of the sample collection unit (20) is connected directly to the inlet (31) of the analysis unit (30) in a liquid-tight manner.

In many diseases it is desirable to diagnose disease conditions as early as possible and/or make an early prognosis of healing progression so as to optionally allow initiation of therapeutic or palliative measures as early as possible.

For example, in the event of bone fractures requiring surgery, a prognosis as to expected healing progression that could be used as a support of early therapeutic decisions is not possible at present. The conventional pattern involves initial surgery of the bone injury. The progression of healing is subsequently monitored over a prolonged period of up to 6 months. Where poor healing is observed, the patients are subjected to further surgery, followed by a suitable treatment with BMP-2 or other factors having a positive influence on the healing process.

One problem arising in this context is that poor healing progression is recognized late, so that treatment thereof as part of a second surgery is late as well.

Frequently, indications for a diagnosis or prognosis of a disease can be obtained using parameters determined from sample material which must be isolated or collected from the patient in question. Conventional devices and methods usually require complex sample processing and/or sample preparation after collecting, so as to allow determination of the desired parameters in subsequent analyses. As a consequence, it is not possible to optionally obtain the results of parameter determination already in the operating room, e.g. during surgery. Rather, the sample material collected from the patient usually must be transmitted from the operating room to a laboratory area where it is processed and subjected to parameter determination. This procedure is lengthy and involves abundant sources of error and possible confusion.

The object of the present invention was to provide means allowing early diagnosis and/or prognosis of a healing process and, in association therewith, an earlier therapy-related decision. More specifically, this is to enable a physician to respond to a prognosis by adapting the treatment even within the time window of surgery.

The present invention solves this problem by providing a device comprising:
i) a sample-receiving unit, said sample-receiving unit comprising a cavity for receiving sample material, means for applying pressure in a regulatable and/or controllable fashion to sample material located in the cavity, and an outlet, said outlet being disposed and designed in such a way that, by actuating the means for applying pressure in a regulatable and/or controllable fashion, components of the sample material can be transferred from the cavity through the outlet and directly to an analysis unit connectable to the outlet;
   and
ii) an analysis unit, said analysis unit comprising an inlet, a first filter, a reservoir and an analysis track, said being inlet designed to be connectable to an outlet of the sample-receiving unit in such a way that components of the sample material can be fed from the sample-receiving unit directly to the analysis unit, said first filter being disposed between inlet and reservoir, so that substantially liquid components of the sample material can be transferred from the inlet through the filter into the reservoir, said reservoir and analysis track being disposed and designed in such a way that filtrate can be transferred from the reservoir to the analysis track;
   wherein the outlet of the sample collection unit is directly connected to the inlet of the analysis unit in a liquid-tight manner.

By operating the means for regulatable and/or controllable application of pressure of the sample receiving unit, sample material situated in the cavity is pressurized, and one or more or all components of the sample material will be conveyed from the cavity toward the outlet. In this way, components of the sample material can be transferred via the outlet to the inlet of an analysis unit directly connected to the sample-receiving unit in a liquid-tight manner. Using the means for regulatable and/or controllable application of pressure, components of the sample material are forced from the inlet of the analysis unit towards the first filter, said first filter allowing passage of substantially liquid components of the sample material into the downstream reservoir, while essentially solid components cannot pass the first filter and therefore cannot enter the reservoir. That is, separation of essentially solid from liquid components of the sample material takes place on the first filter of the analysis unit, said separation being driven by the operation of the means for regulatable and/or controllable application of pressure of the sample-receiving unit and determined by the type and design of the first filter. The filtrate now being situated in the reservoir can be passed to the downstream analysis track. The supply of filtrate from the reservoir into the analysis track may proceed in a passive manner, e.g. as a result of diffusion processes, or actively, e.g. by the means for regulatable and/or controllable application of pressure of the sample-receiving unit. In the analysis track, the filtrate is employed in a detection reaction, the result of which then may optionally be read on or by the analysis track.

One advantage of the inventive device is that, regardless of the consistency of the sample material to be subjected to parameter analysis, rapid and uncomplicated analysis of substantially liquid components of sample material can be performed without requiring complicated sample processing steps. Sample-receiving unit and analysis unit are directly and liquid-tightly connected to each other, so that essentially liquid components of a sample material can be extracted and directly subjected to analysis in a single step. In this way, collection of sample material as well as extraction and analysis thereof can take place on the spot. The use of a plurality of methods and/or devices, possibly spatially separated from each other, is no longer required.

In principle, the device according to the invention can be made of a sterile or sterilizable, especially steam- and/or pressure-sterilizable, material such as metals, alloys or plastics, so that the inventive device is also suitable for direct use in a sterile environment, such as an operating room area.

The device according to the invention, the sample-receiving unit and/or the analysis unit—each one separately or together—can be implemented as a structural unit. A structural unit is understood to be an array of one or more elements which, for the purpose of the intended use, are in a permanently, optionally irreversibly, connected form. In addition, a structural unit may have a housing which surrounds the structural unit.

The device according to the invention is preferably designed and dimensioned in such a way that the device is portable, and in a particularly preferred fashion the device of the invention can be held in one hand.

The sample-receiving unit according to the invention has at least one cavity for receiving sample material, an outlet and means for regulatable and/or controllable application of pressure to sample material located in the cavity. The elements of the sample-receiving unit are designed and arranged in such a way that, by operating the means for applying pressure in a regulatable and/or controllable fashion, components of the sample material can be transferred from the cavity via the outlet and directly to an analysis unit connectable or connected to the outlet.

Sample material is understood to be any biological material that can be collected from an organism or any material containing such biological material, e.g. a tamponade loaded with tissue, cells and/or body fluid. More specifically, the biological material can be tissue, bone, cartilage, tendon and/or cells and/or body fluids such as blood, plasma, serum, synovial fluid, urine, stool, interstitial fluid, lymph, saliva, sweat, spinal fluid and/or lacrimal fluid.

The sample-receiving unit according to the invention may have a base body, preferably a substantially cylindrical base body that surrounds the cavity or wherein the cavity is formed. On a first end of the base body the sample-receiving unit preferably has means for regulatable and/or controllable application of pressure to sample material situated in the cavity, and has an outlet on an end opposite said first end. The cavity is preferably arranged between the means for regulatable and/or controllable application of pressure and the outlet, so that components of the sample material can be conveyed from the cavity to the outlet when operating the means for regulatable and/or controllable application of pressure.

More specifically, the means for regulatable and/or controllable application of pressure on sample material situated in the cavity is designed such that directed pressure can be applied, so that components of the sample material can preferably be conveyed in a particular direction, particularly in the direction of the outlet of the sample-receiving unit. To this end, the means for regulatable and/or controllable application of pressure may comprise a piston that can be pressed into the cavity.

The sample-receiving unit according to the invention can be designed in such a way that sample material, optionally previously obtained (e.g. a tamponade soaked with body fluid), can be introduced into the cavity from the outside. To this end, the sample-receiving unit may have an additional, optionally sealable, access to the cavity, but the sample material can also be introduced into the cavity from the outside through the outlet. In particular, this may apply where the sample-receiving unit is designed such that sample material can be collected from an organism.

In particular, the sample-receiving unit according to the invention can be configured as a press-out unit. For this purpose, the sample-receiving unit on its base body has an opening for loading the cavity with sample material, a means for regulatable and/or controllable application of pressure, e.g. in the form of a piston, and an outlet opposite the means for regulatable and/or controllable application of pressure. Such press-out units are particularly suitable for the use of sample material in the form of a tamponade, which tamponade has previously been placed in a wound and has absorbed liquid components therein. The tamponade is preferably heparinized for sampling. The sample-loaded tamponade can subsequently be supplied to a sample-receiving unit, e.g. in the form of a press-out unit, which, e.g. by pressing out the tamponade, allows supply of components of the sample material to an analysis unit of the invention.

For example, the sample-receiving unit can also be designed as a syringe or suction punch. Preferred sample collection devices, such as syringes or suction punches, allow direct collection of sample material from a human or animal body in such a way that components of the sample material, directly following collection, can be analyzed in an analysis unit directly and liquid-tightly connected or connectable to the sample-receiving unit, without requiring further sample processing steps, possibly in additional containers.

In a particular embodiment the sample collection unit is designed so as to allow collection of a sample at the site of interest, i.e. for example, at the site of injury and/or healing. Sample collection units suitable for this purpose are, for example, syringes and/or punch biopsy devices with/without a vacuum device, e.g. a suction punch.

The analysis unit for use in the device according to the invention has at least one inlet, a first filter, a reservoir and an analysis track. The inlet is designed to be connectable to an outlet of the sample-receiving unit in such a way that components of the sample material can be supplied from the sample-receiving unit directly to the analysis unit. The term “directly” is understood to imply that the outlet of the sample-receiving unit and the inlet of the analysis unit are in direct contact with each other, and there are no means or devices between outlet and inlet other than means for direct, liquid-tight connection, such as pipe-shaped bodies, tubular bodies and/or ring-shaped bodies.

In the analysis unit according to the invention, the first filter is arranged between inlet and reservoir in such a way that essentially liquid components of the sample material can be transferred from the inlet through the filter and into the reservoir. The analysis unit is configured such that transfer of sample material from the inlet into the reservoir is only possible through the first filter. The first filter is designed in such a way that it separates solid from liquid sample constituents and preferably allows passage of essentially liquid components of sample material and substances dissolved therein, such as proteins and hormones. The first filter is preferably selected in such a way to have a pore size allowing passage of at least those substances whose presence is to be determined subsequently in the analysis track in a detection reaction. The selection of the first filter therefore depends on the type and nature of the parameters to be determined in the analysis track and can be determined and established in each case by a person skilled in the art without undue effort. The first filter preferably has a pore size of, for example, ≦10 μm, more preferably ≦5 μm, and especially preferably ≦1 μm. The filter is preferably designed to withstand the pressure required to remove a substantial quantity of essentially liquid components of sample material from the sample and convey it through the first filter into the reservoir.

The analysis unit according to the invention may have one or more additional filters arranged between the inlet and the first filter. These additional second filters can be used to prepare, comminute and/or solubilize the sample material so as to allow transfer of essentially liquid components of the sample material on the first filter as effectively as possible. The above-mentioned additional, second filters preferably have a pore size of ≦200 μm, more preferably ≦150 μm, and especially preferably ≦75 μm.

The first and/or any additional filters are heparinized, if necessary.

Reservoir and analysis track in the analysis unit according to the invention are arranged and designed in such a way that filtrate can be transferred from the reservoir into the analysis track. To this end, the reservoir is positioned between the first filter and the analysis track in the flow direction of the essentially liquid components of the sample material. The analysis unit in the device according to the invention is functionally connected to a sample collection unit in such a way that the essentially liquid components of sample material contained in the cavity of the sample-receiving unit can be transferred through the first filter into the reservoir of the analysis unit; solid components of the sample material are retained by the filter and will not be supplied to the analysis unit. The size of substances present in the filtrate of the sample material is limited by the selection of the first filter. The filter is mounted in the analysis unit in such a way that preferably the transfer of essentially liquid components of the sample from the sample collection device into the reservoir is possible.

The reservoir is designed so as to receive the filtrate following passage through the filter and supply it to the analysis track in such a way that detection of the parameters and/or factors in the filtrate is possible in the analysis track. If necessary, the reservoir has a venting device.

For example, the transfer of substantially liquid components through the first filter can be effected by operating means for regulatable and/or controllable application of pressure to the sample material in the sample-receiving unit. The filter is followed by a reservoir in such a way that the essentially liquid components of the sample material which have passed the first filter are received by said reservoir in the form of a filtrate.

The reservoir may have an additional opening. Said optional opening is provided in addition to the opening for connection to the first filter and the opening for connection to the analysis track. The additional opening can be provided in order to avoid e.g. an overpressure condition in the reservoir during reception of filtrate into the reservoir or create the option of supplying additional liquids or reagents to the filtrate before it enters the analysis track. The additional opening can be designed so as to be sealable, and in a preferred fashion the additional opening is reversibly or irreversibly sealable or sealed. For this purpose, the opening can be provided with, for example, a membrane, a lid and/or a bursting device.

The reservoir is situated between the first filter of the analysis unit and the analysis track. The reservoir is connected to the analysis track in a way so as to allow transfer of filtrate from the reservoir into the analysis track. For example, such transfer may proceed via passive means, e.g. diffusion mechanisms and/or capillary forces. Alternatively or additionally, the transfer can also be mediated actively, e.g. by the means for regulatable and/or controllable application of pressure of the sample-receiving unit.

In the analysis unit according to the invention, the reservoir is followed by the analysis track in such a way that filtrate can be transferred from the reservoir into the analysis track and analyzed there for the presence of one or more specific, predetermined parameters and/or factors. The analysis track is designed so as to allow detection of one or more selected, predetermined parameters in the filtrate from the reservoir. To this end, the analysis track may have a section used to receive filtrate from the reservoir and an optionally separate section functionally connected thereto, which is used to carry out a detection reaction. Depending on the intended detection reaction, selected parameters and/or factors can be determined. For example, this can be the presence or absence of one or more particular substances such as proteins, hormones, nucleic acids and/or other filtrate-soluble substances not excluded by the filter. It is preferred to determine the presence of factors known to be involved in the process of interest, especially a healing process. The analysis track is optionally designed so as to allow detection using well-known detection methods, e.g. test reactions in ELISA format (ELISA=enzyme-linked immunosorbent assay), e.g. by means of an ELISA test strip. In the context of bone injuries it is preferred to use ELISAs for the determination of pro- and antiinflammatory cytokines or proteases and combinations thereof.

If necessary, the analysis track may also comprise control mechanisms by means of which it is possible to determine whether the detection reaction has proceeded properly and specifically. To this end, the analysis track can be designed so as to allow direct reading of the measurement result. In a preferred fashion, no further steps are necessary to allow reading of the measurement result from the outside. For example, the measurement result can be represented in the form of a color reaction.

Reception of the filtrate from the reservoir into the analysis track may proceed in an active or passive manner. Correspondingly, the inventive sample-receiving unit and/or the inventive analysis unit can be configured so as to allow passive and/or active transfer of filtrate from the reservoir into the analysis track. For example, said reception may proceed via passive means, such as means utilizing diffusion mechanisms e.g. across a membrane or a test strip and/or means utilizing capillary forces, etc. Alternatively or additionally, reception may proceed actively, e.g. mediated by the means for regulatable and/or controllable application of pressure of the sample-receiving unit.

The analysis track of the analysis unit according to the invention preferably has a test strip designed and arranged so as to allow passive collection of filtrate from the reservoir by means of the test strip and supply into a detection reaction arranged on said test strip. More specifically, the test strip may have means for a detection reaction in an ELISA format.

The sample-receiving unit and the analysis unit in the device according to the invention are in direct, liquid-tight connection. “Liquid-tight connection” is understood to be any connection that essentially prevents leakage of liquid at the connection between the sample collection unit and the analysis unit during operation of the device according to the invention. To this end, sample-receiving unit and analysis unit can be irreversibly connected to each other, e.g. in the form of a structural unit. Alternatively, sample-receiving unit and analysis unit can be designed so as to be reversibly connected or connectable. “Reversible connection” is understood to be any connection that can be restored liquid-tightly after separation. To this end, the sample-receiving unit and/or analysis unit can have means for reversible, liquid-tight connection, so that the outlet of the sample-receiving unit can be connected in a liquid-tight manner to the inlet of the analysis unit. Such means may comprise means for liquid-tight screw and/or plug connections as well as combinations thereof. Said means preferably comprises means for a liquid-tight screw connection, e.g. a combination of complementary internal and external threads with or without additional interposed seal(s). More specifically, the sample-receiving unit may have an internal thread and the analysis unit an external thread which are complementary to each other and allow formation of a liquid-tight connection. Alternatively, the sample-receiving unit may have an external thread and the analysis unit an internal thread which are complementary to each other and allow formation of a liquid-tight connection.

In addition, the invention discloses a method for early analysis of sample material, said method comprising the steps of:
a) collecting sample material;
b) transferring collected sample material into a cavity of a sample-receiving unit according to the invention;
c) directly transferring liquid components of the sample material from the sample-receiving unit into a reservoir of an analysis unit according to the invention;
d) immediately transferring liquid components of the sample material from the reservoir into the analysis track of the analysis unit using passive and/or active means; and
e) reading the result of analysis from the analysis track of the analysis unit.

The invention also relates to a method of establishing a prognosis of expected healing progression, said method comprising the steps of:
a) obtaining a sample from the tissue of interest;
b) transferring liquid components of the sample into an analysis unit according to the invention;
c) performing the measurement using the analysis unit according to the invention;
d) reading the results of measurement.

In a particular embodiment the method is suitable for establishing a prognosis as to the expected progression of healing of bone injuries, the analysis unit of the invention being configured in such a way that the presence of factors, e.g. pro- and antiinflammatory cytokines and proteases, is measured in the liquid sample components.

The present invention also relates to the above-described sample-receiving unit and/or the above-described analysis unit for use in a device according to the invention.

The invention also relates to a kit including an analysis unit according to the invention, a sample-receiving unit of the invention and/or a device according to the invention.

The inventive analysis unit, sample-receiving unit or device can be used to establish a prognosis as to an expected progression of healing.

The analysis unit according to the invention can be used to determine the presence of factors in healing tissue, especially of pro- and antiinflammatory cytokines and proteases. The healing tissue is preferably tissue obtained during bone surgery, e.g. for the treatment of a bone fracture or insertion of an endoprosthesis.

By virtue of the invention, it is possible for the first time to make an early decision, even during surgery, as to therapy including BMP-2 administration in cases of bone injuries instead of a decision as late as after a 6-month follow-up period as otherwise usual.

The invention allows collecting a suitable sample already in the operating room and establishing a prognosis as to expected progression of healing by determining the presence of suitable factors, such as BMP-2, MMP-2 and/or VEGF, thereby providing assistance in reaching a decision as to early planning an appropriate therapy, optionally a therapy including BMP-2 administration.

The new method allows prognosis as early as during initial surgery, so that appropriate treatment can follow immediately. A second surgery for treatment can thus be avoided.

One particular advantage of devices including an inventive analysis unit and a sample collection device, wherein the analysis unit is functionally connected to the sample collection device so as to allow transfer of liquid sample components from the sample collection device into the analysis unit, is that the sample need not be transferred into another container after collecting and until the result is provided.

The present invention also relates to:
An analysis unit designed so as to be surface-mountable on or functionally connectable to a sample collection device, said analysis unit comprising at least one filter, a reservoir and an analysis track.
A device comprising said analysis unit and a sample collection device, said analysis unit being functionally connected to the sample collection device in such a way that liquid sample components can be transferred from the sample collection device into the analysis unit.
A kit including said analysis unit or said device.
A method of establishing a prognosis as to an expected progression of healing, said method comprising the steps of:
a) obtaining a sample from the tissue of interest;
b) transferring liquid components of the sample into said analysis unit;
c) performing the measurement using said analysis unit;
d) reading the results of measurement.
   Use of said analysis unit or said device or said kit in establishing a prognosis as to an expected progression of healing.

### FIGURES

FIG. 1 shows a schematic representation of a device according to the invention, wherein sample-receiving unit and analysis unit are irreversibly connected to each other.

FIG. 2 schematically shows the operation of the device of FIG. 1 in FIGS. 2 A, B and C.

FIG. 3 shows a schematic representation of an embodiment of a sample-receiving unit in the form of a press-out unit.

FIG. 4 shows a sample-receiving unit in the form of a suction punch in A, and reception of sample material in the suction punch is schematically shown in B.

FIG. 5 schematically shows in FIGS. 5 A, B and C the operation of a device consisting of a sampling unit from FIG. 4 and an analysis unit.

FIG. 6 shows a sample-receiving unit in the form of a suction punch in overall view (A), sectional view (B) and detail view (C).

### REFERENCE LIST

1 Device comprising sample-receiving unit and analysis unit
20 Sample-receiving unit
21 Base body
22 Piston
23 Opening
24 Cavity
25 Outlet
26 Thread
27 Outlet end with cutting edge
28 Trigger
28a Grid for locking
29 Return spring
30 Analysis unit
31 Inlet
32 Filter
33 Reservoir
34 Opening
35 Analysis track
36 Seal
37 Thread
40 Sample material
41 Filtrate

The invention will be illustrated in more detail in the examples below.

### EXAMPLE 1

### First Embodiment of a Device of the Invention

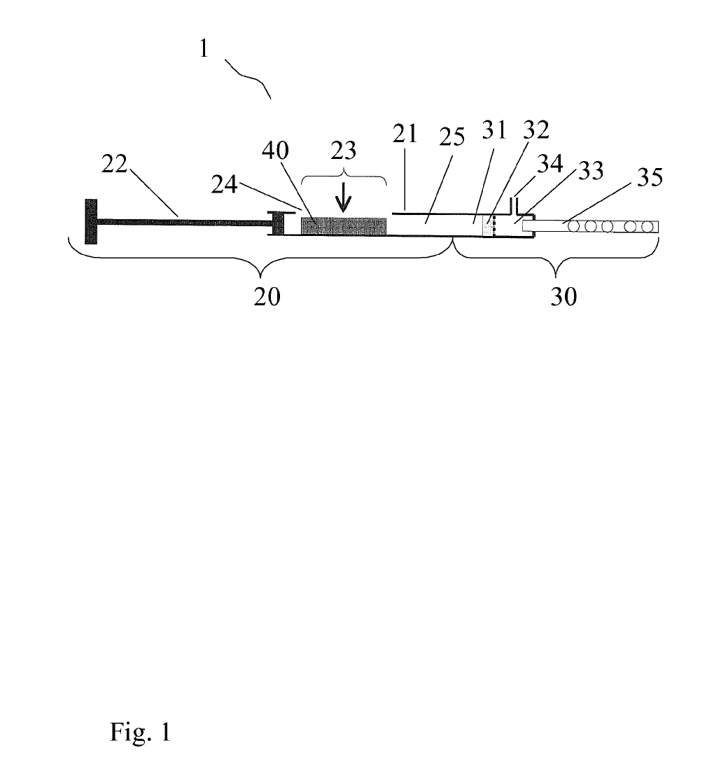

A first embodiment of a device of the invention having sample material arranged in the cavity is schematically shown in FIG. 1. It is a device 1 wherein sample-receiving unit 20 and analysis unit 30 are irreversibly connected to each other in the form of a structural unit. The sample-receiving unit 20 has a substantially cylindrical base body 21 wherein a cavity 24 for receiving sample material 40 is accessible via an opening 23. On a first end of base body 21, the sample-receiving unit 20 has a piston 22 for regulatable and/or controllable application of pressure to sample material 40 situated in the cavity 24. At the end of base body 21 opposite the piston 22 the sample-receiving unit 20 has an outlet 25 in direct and liquid-tight connection to the analysis unit 30. The outlet 25 is directly followed by the inlet 31 of analysis unit 30 in irreversible, liquid-tight connection. The first filter 32 is arranged between inlet 31 and reservoir 33 of the analysis unit 30, so that sample material can only enter the reservoir 33 from the inlet 31 through the first filter 32. The reservoir 33 has an additional opening 34. The reservoir 33 is followed by the analysis track 35.

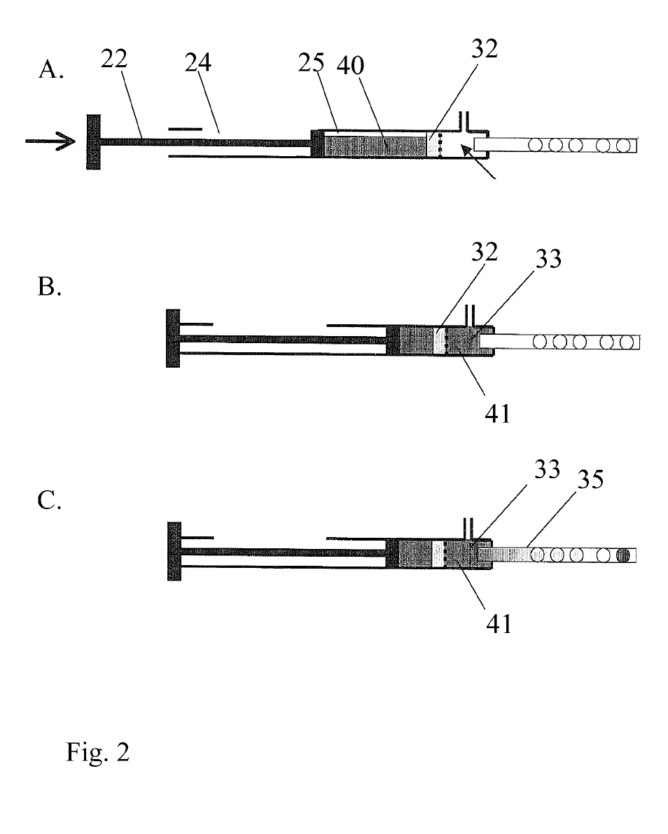

The operation of device 1 is schematically shown in FIGS. 2 A, B and C. Once sample material 40 in the form of a tamponade loaded with body fluid has been received in cavity 24 through opening 23 of the sample-receiving unit 20, pressure is applied to the sample material 40 by forcing the piston 22 inwardly. By forcing the piston 22 inwardly, sample material 40 is conveyed from cavity 24 via outlet 25 out of the sample-receiving unit 20 and supplied to the analysis unit 30 through the inlet 31, as shown in FIG. 2 A. In the analysis unit 30 the sample material 40 contacts the filter 32, and as a result of the pressure applied to the sample material 40 by forcing the piston 22 inwardly, essentially liquid components of the sample material 40 are forced through the filter 32 in the form of a filtrate 41. As shown in FIG. 2 B, the filtrate 41 accumulates in reservoir 33 of the analysis unit, so that the sample-receiving zone of analysis track 35 is now in contact with the filtrate 41 to receive the latter. As shown in FIG. 2 C, the filtrate 41 from reservoir 33 is received by the analysis track 35 to contact a zone of the analysis track which has a detection reaction in an ELISA format coated thereon. As soon as the filtrate 41 makes contact with the detection zone of analysis track 35, the detection reaction is triggered and, after the detection reaction is completed, the test result can be read from the outside as a colored label on analysis track 35.

### EXAMPLE 2

### Sample-Receiving Unit in the Form of a Press-Out Unit

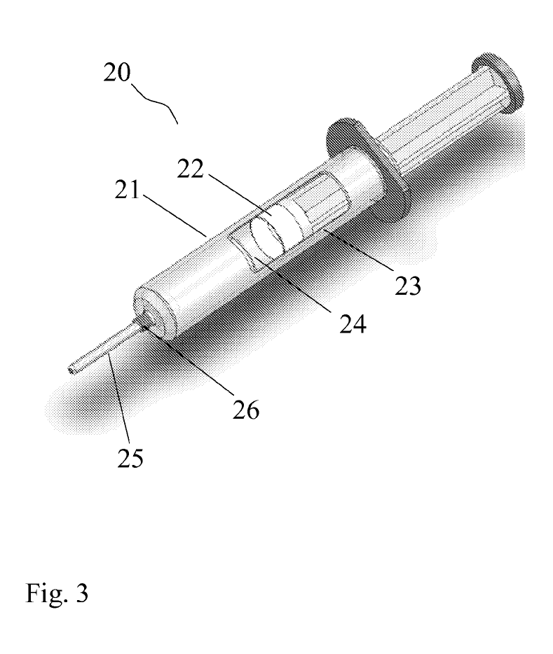

FIG. 3 schematically represents an embodiment of a sample-receiving unit 20 in the form of a press-out unit. The press-out unit 20 has a substantially cylindrical body 21 with an opening 23 through which a cavity 24 for receiving sample material is accessible. On a first end of base body 21, the press-out unit 20 has means for regulatable and/or controllable application of pressure to sample material situated in cavity 24, which means is in the form of a piston 22 that can be forced into cavity 24. At the end of base body 21 opposite the first end the press-out unit 20 has an outlet 25 through which components of the sample material, by actuating the piston 22, can be transferred directly from cavity 24 through outlet 25 to an analysis unit connectable to the outlet 25. At the end of base body 21 where the outlet 25 is situated, the press-out unit 20 has means for reversible, liquid-tight connection to an analysis unit in the form of a thread 26 for a screw connection.

For example, the press-out unit 20 can be used to squeeze out a tamponade loaded with body fluid and directly supply components of the sample material to an analysis unit connected to the press-out unit 20.

### EXAMPLE 3

### Second Embodiment of a Device of the Invention

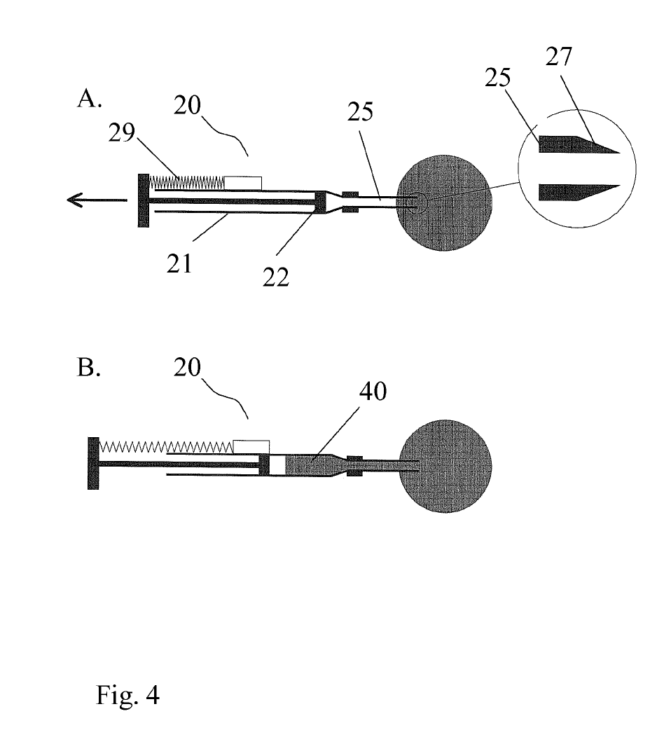

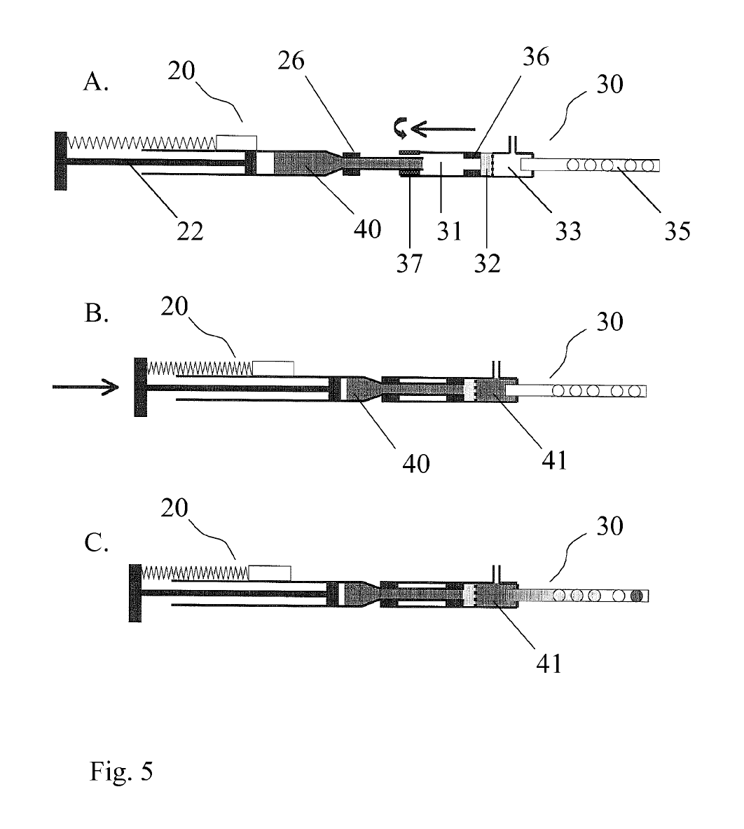

FIGS. 4 A and B schematically represent a sample-receiving unit 20 in the form of a suction punch and the reception of sample material. The sample-receiving unit 20 in the form of a suction punch has a substantially cylindrical base body 21, a cavity 24 for receiving sample material, a piston 22 driven by a preloaded, lockable return spring 29, a thread 26 and an outlet opening 25 having sharpened cutting edges at its end 27. The sample-receiving unit 20 in the form of a suction punch can be loaded with sample material by introducing the outlet end 27 into the biological material of interest and triggering the piston 22 by releasing the locked return spring 29. In this way, the piston is moved away from the outlet end to generate a vacuum as a result of which sample material is received in cavity 24 of the suction punch through outlet 25. At this point, the sample-receiving unit 20 in the form of a suction punch is loaded with sample material 40. As shown in FIG. 5 A, the inventive device 1 is now assembled from the sample-receiving unit 20 in the form of a suction punch and an analysis unit 30 by reversible, liquid-tight, direct screw connection via the complementary threaded parts 26 of the sample-receiving unit and 37 of the analysis unit. The sample material 40 is subsequently transferred into the analysis unit 30 by forcing the piston 22 inwardly, and the filtrate 41 enters the reservoir 33, as shown in FIG. 5 B. Thereafter, the filtrate 41 is introduced into the analysis track 35, and after completed detection reaction the result of measurement can be read in the form of a color reaction on analysis track 35 (see FIG. 5 C).

### EXAMPLE 4

### Sample-Receiving Unit in the Form of a Suction Punch

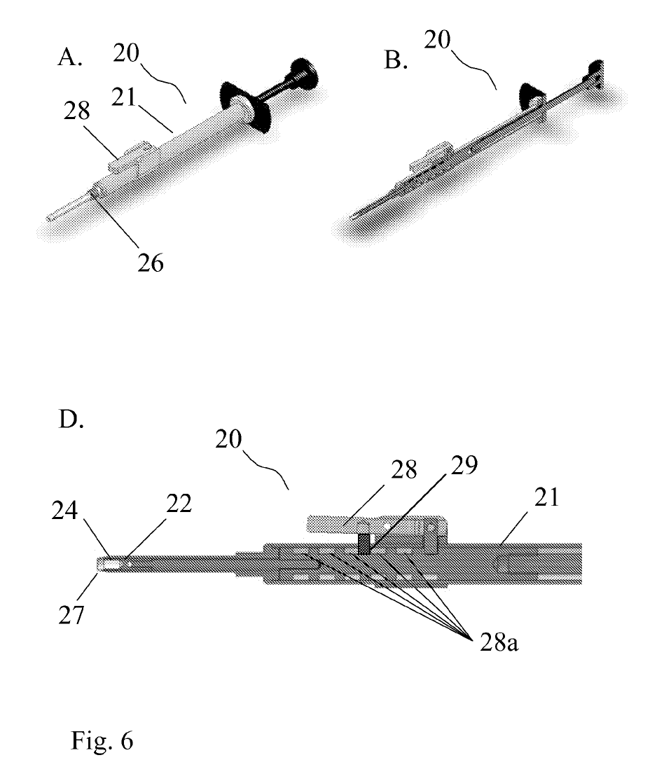

FIG. 6 schematically shows an embodiment of a sample-receiving unit 20 in the form of a suction punch in overall view in FIG. 6 A, in sectional view in FIG. 6 B, and in detail view in FIG. 6 C.

The sample-receiving unit 20 in the form of a suction punch has a substantially cylindrical base body 21. On a first end of the base body 21 the sample-receiving unit 20 in the form of a suction punch has a piston 22 with a return spring 29 (not shown) and a trigger 28, said trigger being locked on a grid 28a and preloaded by means of a spring or a cylinder made of elastic material 29. At the end of base body 21 opposite the first end the sample-receiving unit 20 in the form of a suction punch has an outlet having an outlet end 27 with a cutting edge. At the end of base body 21 where the outlet 27 is situated, the sample-receiving unit 20 in the form of a suction punch has means for reversible, liquid-tight connection to an analysis unit in the form of a thread 26 for a screw connection. When the trigger 28 is actuated, the return spring 29 causes the piston 22 to move away from the outlet end 27. A vacuum is generated, and reception of sample material into the cavity 24 of the suction punch takes place. If the suction punch is in direct, liquid-tight connection to an analysis unit via thread 26, sample material can be transferred from the suction punch into the analysis unit by forcing the piston 22 inwardly.

1. A device comprising:
i) a sample-receiving unit (20), said sample-receiving unit (20) comprising a cavity (24) for receiving sample material, means (22) for applying pressure in a regulatable and/or controllable fashion to a sample material located in the cavity, and an outlet (25), said outlet (25) being disposed and designed in such a way that, by actuating the means (22) for applying pressure in a regulatable and/or controllable fashion, components of the sample material can be transferred from the cavity (24) through the outlet (25) and directly to an analysis unit (30) connectable to the outlet (25);
and
ii) an analysis unit (30), said analysis unit (30) comprising an inlet (31), a first filter (32), a reservoir (33) and an analysis track (35), said inlet (31) being designed to be connectable to an outlet (25) of the sample-receiving unit (20) such that components of the sample material can be fed from the sample-receiving unit (20) directly to the analysis unit (30), said first filter (32) being disposed between inlet (31) and reservoir (33), so that substantially liquid components of the sample material can be transferred from the inlet (31) through the filter (32) into the reservoir (33), said reservoir (33) and analysis track (35) being disposed and designed in such a way that filtrate can be transferred from the reservoir (33) to the analysis track (35);
wherein the outlet (25) of the sample receiving unit (20) is directly connected to the inlet (31) of the analysis unit (30) in a liquid-tight manner.

2. The device as claimed in claim 1, wherein the sample-receiving unit (20) and/or the analysis unit (30) are designed in such a way that filtrate can be transferred passively and/or actively from the reservoir (33) into the analysis track (35).

3. The device as claimed in claim 1, wherein the sample-receiving unit (20) and the analysis unit (30) are irreversibly connected to each other.

4. The device as claimed in claim 1, wherein the sample-receiving unit (20) and the analysis unit (30) are designed so as to be reversibly connected or connectable.

5. The device as claimed in claim 4, wherein the sample-receiving unit (20) and/or analysis unit (30) have means for a reversible, liquid-tight connection of the outlet (25) of sample-receiving unit (20) to the inlet (31) of analysis unit (30).

6. The device as claimed in claim 5, wherein the means allow liquid-tight screw and/or plug connection.

7. The device as claimed in claim 1, wherein the reservoir (33) of the analysis unit (30) has an additional opening (34).

8. The device as claimed in claim 1, wherein the first filter (32) of the analysis unit (30) has a pore size of ≦10 μm, preferably ≦5 μm, more preferably ≦1 μm.

9. The device as claimed in claim 1, wherein the analysis unit (30) has a second filter which is arranged between the inlet (31) and the first filter (32) of the analysis unit (30) and has a pore size of ≦200 μm, preferably ≦150 μm, more preferably ≦75 μm.

10. The device as claimed in claim 1, wherein the analysis track (35) of the analysis unit (30) has a test strip designed and arranged so as to allow passive collection of filtrate from the reservoir (33) by means of said test strip and supply into a detection reaction arranged on said test strip.

11. The device as claimed in claim 10, wherein the test strip has means for a detection reaction in an ELISA format.

12. The device as claimed in claim 1, wherein the cavity (24) of the sample-receiving unit (20) is designed in such a way that sample material previously obtained can be introduced into the cavity (24) from the outside.

13. The device as claimed in claim 1, wherein the sample-receiving unit (20) is designed such that sample material can be collected from an organism.

14. The device as claimed in claim 1, wherein the sample-receiving unit (20) has a substantially cylindrical base body (21) that surrounds the cavity (24).

15. The device as claimed in claim 1, wherein the means (22) for regulatable and/or controllable application of pressure comprises a piston that can be forced into the cavity.

16. The device as claimed in claim 1, wherein the sample-receiving unit (20) is designed in the form of a syringe, press-out unit or suction punch.

17. The device as claimed in claim 1, wherein the device is designed so as to be portable and preferably can be held in one hand.

18. The device as claimed in claim 1, wherein the sample-receiving unit (20), analysis unit (30) or the entire device are implemented in the form of a structural unit.

19. (canceled)

20. (canceled)

21. A method for early analysis of sample material, said method comprising the steps of:
a) collecting sample material;
b) transferring collected sample material into a cavity (24) of a sample-receiving unit (20) according to claim 18;
c) directly transferring liquid components of the sample material from the sample-receiving unit (20) into a reservoir (33) of an analysis unit (30) according to claim 19;
d) immediately transferring liquid components of the sample material from the reservoir (33) into the analysis track (35) of the analysis unit using passive and/or active means; and
e) reading the result of analysis from the analysis track (35) of the analysis unit (30).
